(19) United States
(12) Patent Application Publication
(10) Pub. No.: US 2006/0172904 A1
(43) Pub. Date: Aug. 3, 2006

(54) Makeup-removing composition

(75) Inventor: Arnaud Bonafos, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/302,246

(22) Filed: Dec. 14, 2005

Related U.S. Application Data
(60) Provisional application No. 60/672,864, filed on Apr. 20, 2005.

(30) Foreign Application Priority Data
Jan. 31, 2005 (FR) 05 50263

(51) Int. Cl. A61K 8/00

(52) U.S. Cl. 510/130; 510/438; 510/424

(57) ABSTRACT

The invention relates to a composition in the form of an oil-in-water emulsion containing at least one silicone surfactant, at least one oil, and at least one detergent surfactant. In a preferred embodiment the composition contains no other emulsifier other than the at least one silicone surfactant. The addition of the silicone surfactant makes it possible to notably improve the makeup-removing capacity of the composition while at the same time leaving no greasy or tacky feeling on the skin. The composition may preferably be in the form of a milk or of a cream.

### REFERENCE TO PRIOR APPLICATIONS

This application claims priority to U.S. provisional application 60/672,864 filed Apr. 20, 2005, and to French patent application 0550263 filed Jan. 31, 2005, both incorporated herein by reference.

### FIELD OF THE INVENTION

The present invention relates to a composition in the form of an oil-in-water (o/W) emulsion, comprising a silicone surfactant, and to the use of this composition for cleansing or removing makeup from the skin of the face and/or of the body. In particular, this emulsion can constitute a makeup-removing milk or a makeup-removing cream.

The invention also relates to a process for cleansing or removing makeup from the skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The products for removing makeup from and cleansing the face that are in the form of emulsions, conventionally used today, have drawbacks that are not the same depending on the nature of the emulsion. In the case of water-in-oil (W/o) emulsions, which are very effective for makeup removal/cleansing because the external fatty phase is directly available for dissolving the various fatty substances present on the skin or derived from the makeup, their use is uncomfortable due to the greasy and heavy feeling afforded by this external fatty phase which remains on the skin.

In the case of oil-in-water (O/W) emulsions, the external aqueous phase provides freshness. On the other hand, since the oily phase is the internal phase, it is not directly available for dissolving the fatty substances and, as a result, the makeup removal is less efficient. Now, for cleansing the face, and more especially for makeup removal, which can consist in removing all the makeup products, women seek to obtain makeup removal that is as efficient as possible.

In order to improve the efficiency of a makeup-removing composition in the form of an O/W emulsion, it is necessary to introduce a large amount of fatty phase (oily phase), since it is the latter that ensures good dissolution of the solid particles of which the makeup is composed. However, when a large amount of oil is introduced into such a composition, the cosmetic qualities and the tolerance of the composition are compromised, since compositions containing too large an amount of oil, for example 30%, leave a greasy film on the skin and give the skin a shiny and tacky appearance, which is often unacceptable to the user.

There remains therefore the need for a makeup-removing emulsion that is very efficient while at the same time being well tolerated and having good cosmetic properties, i.e. having neither a greasy effect nor a tacky effect nor a shiny effect.

### SUMMARY OF THE INVENTION

The inventor has found, surprisingly, that it is possible to obtain a makeup-removing composition in the form of an O/W emulsion containing a large amount of oil, using a silicone surfactant.

One subject of the present invention is therefore a composition in the form of an oil-in-water emulsion, comprising an oily phase dispersed in an aqueous phase, wherein the composition comprises at least one silicone surfactant, at least one oil, and at least one detergent surfactant.

In preferred embodiments, the invention composition is a cosmetic composition and/or constitutes in particular a cleansing or makeup-removing composition.

The emulsion according to the invention also preferably has the property not only being non-irritating and stable, but also of being very efficient while at the same time being pleasant to use, in particular being very soft when applied, and leaving no greasy or tacky feeling. These properties are very important in this field, and are properties that greatly affect acceptance in the marketplace and the overall success of a product.

### DETAILED DESCRIPTION OF THE PREFERRED EMBODIMENTS

The inventor has noted, surprisingly, that silicone surfactants increase the makeup-removing efficiency of oils present in O/W emulsions. The result obtained is all the more surprising since silicone surfactants are known to be emulsifiers of W/o emulsions, whereas, here, it is an O/W emulsion and the emulsion preferably contains no other emulsifier—the other surfactants that it contains are detergent surfactants and not emulsifying surfactants.

Thus, one subject of the present invention is the use of at least one silicone surfactant for improving the makeup-removing efficiency of an oil-in-water emulsion containing at least one oil and at least one detergent surfactant.

Since the invention composition is a composition typically intended for topical application, it preferably comprises a physiological acceptable medium. In the present application, the term “physiologically acceptable medium” is intended to mean a medium that is compatible with all keratin materials, such as the skin, including the scalp, the nails, the mucous membranes, the eyes and the hair, or any other cutaneous area of the body.

Moreover, the term “detergent surfactant” is intended to mean a surfactant that is incapable of stabilizing, on its own in the long term, an emulsion, but that has detergent properties, i.e. able to give a cleansing or detergent effect. Generally, such surfactants have quite a high HLB (hydrophilic lipophilic balance). The term “HLB” is well known to those skilled in the art and is described in “The HLB system. A time-saving guide to Emulsifier Selection” (published by ICI Americas Inc: 1984). Preferably, the detergent surfactant used or the mixture of detergent surfactants used has an HLB greater than or equal to 11, for example ranging from 11 to 18, and preferably from 12 to 17.

Silicone Surfactant

Useful silicone surfactants are emulsifying surfactants and they include polydimethylsiloxanes comprising oxyethylenated and/or oxypropylenated groups. Use is preferably made of those comprising an oxyethylenated and/or oxypropylenated chain as shown below:
—(C₂H₄O)ₓ—(C₃H₆O)_{y}—H
where x can range from 0 to 100 and y can range from 0 to 100, it being impossible for x and y to both be 0.

According to a preferred embodiment of the invention, these silicone surfactants comprise both oxyethylenated groups and oxypropylenated groups, for example from 2 to 50 oxyethylenated groups and from 2 to 50 oxypropylenated groups, each range of 2-50 including 5, 10, 20, 30, and 40, as well as the subrange 16-20. The silicone surfactants used in the composition of the invention can be chosen from, for example, dimethicone copolyols and alkyl dimethicone copolyols.

The dimethicone copolyols that can be used in the composition of the invention include in particular those comprising from 2 to 50 oxyethylenated groups and from 2 to 50 oxypropylenated groups, for instance that comprising 18 oxyethylenated groups and 18 oxypropylenated groups (PEG/PPG-18/18 dimethicone), such as the mixture of dimethicone copolyol comprising 18 oxyethylenated groups and 18 oxypropylenated groups, of cyclopentasiloxane and of water (10/88/2) sold by the company Dow Corning under the name DC-3225C or DC2-5225C (INCI name: cyclopentasiloxane/PEG/PPG-18/18 dimethicone), or comprising 14 oxyethylenated groups and 14 oxypropylenated groups (PEG/PPG-14/14 dimethicone) such as, in particular, the mixture of dimethicone copolyol comprising 14 oxyethylenated groups and 14 oxypropylenated groups and of cyclopentasiloxane (85/15) sold under the name Abil EM-97 by the company Goldschmidt (INCI name: Bis-PEG/PPG-14/14 dimethicone/cyclopentasiloxane).

As alkyl dimethicone copolyols, mention may in particular be made of those having an alkyl radical comprising from 10 to 22 carbon atoms and comprising from 2 to 50 oxyethylenated groups and from −2 to 50 oxypropylenated groups, such as cetyl dimethicone copolyol (INCI name: Cetyl PEG/PPG-10/1 dimethicone), for instance the product sold under the name Abil EM-90 by the company Goldschmidt; or lauryl dimethicone copolyol (INCI name: Lauryl PEG/PPG-18/18 methicone) and, for example, the mixture of approximately 91% of lauryl dimethicone copolyol and of approximately 9% of isostearyl alcohol sold under the name Dow Corning 5200 Formulation Aid by the company Dow Corning.

A mixture of these silicone surfactants may also be used.

According to a particularly preferred embodiment, the silicone surfactant is chosen from dimethicone copolyols, and in particular that comprising 18 oxyethylenated groups and 18 oxypropylenated groups, more particularly the product sold under the name DC-5225 C.

The amount of silicone surfactant in the composition of the invention, with respect to active material, is not particularly limited and may range, for example, from 0.01 to 5% by weight, preferably from 0.05 to 3% by weight, and better still from 0.05 to 2% by weight relative to the total weight of the composition.

Oily Phase

The amount of oily phase present is not particularly limited and may constitute from 3 to 70% by weight, preferably from 5 to 60% by weight, and better still from 10 to 50% by weight, relative to the total weight of the composition.

The oily phase contains the oils and other fatty substances and the lipophilic additives. It preferably contains at least one oil, and in particular at least one makeup-removing oil chosen from hydrocarbon-based oils, fatty esters comprising at least 8 carbon atoms, and mixtures thereof.

The term “hydrocarbon-based oil” is intended to mean any oil comprising predominantly carbon and hydrogen atoms. As hydrocarbon-based oils, mention may, for example, be made of volatile or non-volatile paraffin oils, and derivatives thereof, liquid petroleum jelly, polydecenes, hydrogenated polyisobutene such as that sold under the name Parleam® oil, hydrogenated isoparaffin, isohexadecane and isododecane, and mixtures thereof.

The term “fatty ester” is intended to mean an ester obtained from a fatty acid and/or from a fatty alcohol. The fatty esters comprise at least 8 carbon atoms, and preferably from 9 to 26 carbon atoms, and better still from 12 to 22 carbon atoms. The ester constituting the makeup-removing oil may particularly be chosen from:
(1) esters obtained from a straight or branched chain fatty acid comprising at least 9 carbon atoms and from a straight or branched chain alcohol comprising from 1 to 17 carbon atoms;
(2) esters obtained from a straight or branched chain acid comprising 1 to 17 carbon atoms and from a straight or branched chain fatty alcohol comprising at least 9 carbon atoms;
(3) esters obtained from benzoic acid and from a straight or branched chain fatty alcohol comprising at least 9 carbon atoms; and
(4) mixtures thereof.

The esters are preferably monoesters or diesters.

Preferably, the ester that may constitute the makeup-removing oil is chosen from the group of esters that have no unsaturation and/or no ether or hydroxyl group. Even more advantageously, it is a saturated ester that contains no ether or hydroxyl group.

Thus, the ester that may be used as a makeup-removing oil in the composition in accordance with the invention can in particular be chosen from the group comprising 2-ethylhexyl palmitate (or octyl palmitate), isononyl isononanoate, isostearyl lactate, 2-ethylhexyl myristate (or octyl myristate), isopropyl palmitate, isopropyl myristate, diisopropyl adipate, dioctyl adipate, 2-ethylhexyl hexanoate, ethyl laurate, methyl myristate, octyldodecyl octanoate, isodecyl neopentanoate, ethyl myristate, myristyl propionate, 2-ethylhexyl 2-ethyl hexanoate, 2-ethylhexyl octanoate, 2-ethylhexyl caprate/caprylate, methyl palmitate, butyl myristate, isobutyl myristate, ethyl palmitate, isohexyl laurate, hexyl laurate, isopropyl isostearate, the ester of benzoic acid and of C₁₂-C₁₅ alcohols (INCI name: C₁₂-C₁₅ alkyl benzoate), and mixtures thereof.

The makeup-removing oil(s) may constitute all or part of the oily phase and, for example, the amount of makeup-removing oil(s) may range from 0.5 to 70%, preferably 2 to 60%, and better still from 5 to 50% by weight, relative to the total weight of the composition.

The oily phase may also contain any of the fatty substances, and in particular the oils, other than those indicated above, conventionally used in the cosmetic field. As other oils that may be present in the oily phase, mention may, for example, be made of oils of plant origin, such as apricot kernel oil or sweet almond oil; synthetic oils; volatile or non-volatile silicone oils and fluoro oils.

As synthetic oils, mention may, for example, be made of Purcellin oil; polyol esters, such as propylene glycol dioctanoate, neopentyl glycol diheptanoate and diethylene glycol diisononanoate; pentaerythritol esters such as pentaerythrityl tetraisostearate; lipophilic derivatives of amino acids, such as isopropyl lauroyl sarcosinate.

As silicone oils, mention may, for example, be made of volatile or non-volatile polydimethylsiloxanes (PDMS) comprising a linear or cyclic silicone chain which are liquid or pasty at ambient temperature, in particular cyclopolydimethylsiloxanes (cyclomethicones), such as cyclohexasiloxane and cyclopentasiloxane; polydimethylsiloxanes comprising pendant alkyl, alkoxy or phenyl groups or alkyl, alkoxy or phenyl groups at the silicone chain end, which groups have from 2 to 24 carbon atoms; phenylated silicones, such as phenyl trimethicones, phenyl dimethicones, phenyltirmethylsiloxydiphenylsiloxanes, diphenyl dimethicones, diphenylmethyldiphenyltrisoloxanes, (2-phenylethyl)trimethylsiloxysilicates and polymethylphenylsiloxanes; mixtures thereof.

The other fatty substances that can be present in the oily phase may, for example, be fatty acids, fatty alcohols such as cetyl alcohol, stearyl alcohol and mixtures thereof (cetearyl alcohol), petroleum jelly (petrolatum) and waxes.

Aqueous Phase

The composition according to the invention comprises an amount of aqueous phase that is not particularly limited and that may range, for example, from 30 to 97% by weight, preferably from 40 to 95% by weight, and better still from 50 to 90% by weight, relative to the total weight of the composition.

The aqueous phase comprises water and any other water-soluble compound(s) optionally present, such as in particular water-soluble solvents and additives (for example, hydrophilic surfactants and active agents).

As water-soluble solvents, mention may in particular be made of lower alcohols and polyols. The term “lower alcohol” is intended to mean alcohols comprising from 1 to 8, and more particularly from 1 to 6, carbon atoms, such as ethanol. As polyols, mention may, for example, be made of glycerol, propylene glycol, butylene glycol and polyethylene glycols (PEG-8, for example). These alcohols and/or polyols may be present in the composition in any amount such as an amount preferably ranging from 0.1 to 25%, and from 1 to 15%, of the total weight of the composition.

Detergent Surfactants

While not bound by a particular theory, it is believed that the detergent surfactants are what give the composition its cleansing nature and/or improve it. The surfactants can be chosen from nonionic, anionic, amphoteric and zwitterionic detergent surfactants, and mixtures thereof. As indicated above, the detergent surfactants used or the mixture of detergent surfactants used preferably generally has an HLB (hydrophilic lipophilic balance) greater than or equal to 11.

The amount of detergent surfactant(s) is not limited and may range, for example, in terms of weight of active material, from 0.05 to 10% by weight, preferably from 0.1 to 5% by weight, and better still from 0.2 to 2% by weight, relative to the total weight of the composition.

As detergent surfactants, mention may be made of:

1. Nonionic Surfactants:

As nonionic surfactants, use may, for example, be made of alkyl polyglucosides (APGs), maltose esters, polyglycerolated fatty alcohols, glucamine derivatives such as 2-ethylhexyloxycarbonyl-n-methyl glucamine, and mixtures thereof.

As alkylpolyglucosides, use is preferably made of those containing a straight chain alkyl group comprising from 6 to 30 carbon atoms, and preferably from 8 to 16 carbon atoms, and containing a hydrophilic group (glucoside) preferably comprising 1.2 to 3 glucoside units. As alkylpolyglucosides, mention may, for example, be made of decylglucoside (alkyl-C9/C11-polyglucoside (1.4)), for instance the product sold under the name Mydol 10® by the company Kao Chemicals, the product sold under the name Plantaren 2000 UP® by the company Cognis, and the product sold under the name Oramix NS 10® by the company Seppic; caprylyl/capryl glucoside, for instance the product sold under the name Oramix CG 110® by the company Seppic; laurylglucoside, for instance the products sold under the names Plantaren 1200 N® and Plantacare 120° by the company Cognis; and cocoglucoside, for instance the product sold under the name Plantacare 818/UP® by the company Cognis.

The maltose derivatives are, for example, those described in document EP-A-566438, such as O-octanoyl-6′-D-maltose or O-dodecanoyl-6′-D-maltose described in document FR-2,739,556.

Among the polyglycerolated fatty alcohols, mention may be made of polyglycerolated dodecanediol (3.5 mol of glycerol), a product sold under the name Chimexane NF® by the company Chimex.

2. Anionic Surfactants:

These surfactants may be chosen in particular from carboxylates, amino acid derivatives, alkyl sulphates, alkyl ether sulphates, sulphonates, isethionates, taurates, sulphosuccinates, alkyl sulphoacetates, phosphates and alkyl phosphates, polypeptides, anionic derivatives of alkyl polyglucoside, fatty acid soaps, and mixtures thereof.

As carboxylates, mention may, for example, be made of alkali metal salts of N-acylamino acids; amido ether carboxylates (AEC), for instance sodium laurylamido ether carboxylate (3 EO) sold under the name Akypo Foam 30® by the company Kao Chemicals; polyoxyethylenated carboxylic acid salts, for instance oxyethylenated (6 EO) sodium lauryl ether carboxylate (65/25/10 C12-14-16) sold under the name Akypo Soft 45 NV® by the company Kao Chemicals; polyoxyethylenated fatty acids of olive oil and of carboxymethyl, product sold under the name Olivem 400® by the company Biologia E Tecnologia; oxyethylenated (6 EO) sodium tridecyl ether carboxylate sold under the name Nikkol ECTD-6NEX® by the company Nikkol.

The amino acid derivatives may be chosen, for example, from sarcosinates, and in particular acyl-sarcosinates, such as sodium lauroyl sarcosinate sold under the name Sarkosyl NL 97® by the company Ciba or sold under the name Oramix L 30® by the company Seppic, sodium myristoyl sarcosinate sold under the name Nikkol Sarcosinate MN® by the company Nikkol, sodium palmitoyl sarcosinate sold under the name Nikkol Sarcosinate PN® by the company Nikkol; alaninates, for instance sodium N-lauroyl-N-methylamidopropionate sold under the name Sodium Nikkol Alaninate LN 30® by the company Nikkol or sold under the name Alanone ALE® by the company Kawaken, and the N-lauroyl N-methylalanine triethanolamine sold under the name Alanone Alta® by the company Kawaken; N-acylglutamates, for instance the triethanolamine mono-cocoylglutamate sold under the name Acylglutamate CT-12 by the company Ajinomoto, and the triethanolamine lauroylglutamate sold under the name Acylglutamate LT-12® by the company Ajinomoto; aspartates, for instance the mixture of triethanolamine N-lauroylaspartate and of triethanolamine N-myristoylaspartate sold under the name Asparack® by the company Mitsubishi; citrates, and mixtures thereof.

As alkyl sulphates, mention may, for example, be made of triethanolamine lauryl sulphate (INCI name: TEA-lauryl sulphate) such as the product sold by the company Huntsman under the name Empicol TL40 FL or that sold by the company Cognis under the name Texapon T42, products which are at 40% in aqueous solution. Mention may also be made of ammonium lauryl sulphate (CFTA name: ammonium lauryl sulphate) such as the product sold by the company Huntsman under the name Empicol AL 30FL, which is at 30% in aqueous solution.

As alkyl ether sulphates, mention may, for example, be made of sodium lauryl ether sulphate (INCI name: sodium laureth sulphate), for instance that sold under the names Texapon N 40 and Texapon AOS 225 UP by the company Cognis, and ammonium lauryl ether sulphate (INCI name: ammonium laureth sulphate), for instance that sold under the name Standapol EA-2 by the company Cognis.

As sulphonates, mention may, for example, be made of alpha-olefin sulphonates, for instance the sodium alpha-olefin sulphonate (C14-16) sold under the name Bio-terge AS-40® and Bio-terge AS-40 CG® by the company Stepan, or under the names Witconate AOS Protége and Sulframine AOS PH 12® by the company Witco; the sodium secondary olefin sulphonate sold under the name Hostapur SAS 30® by the company Clariant; linear alkyl aryl sulphonates, for instance the sodium xylene sulphonate sold under the names Manrosol SXS30®, Manrosol SXS40® and Manrosol SXS93® by the company Manro.

As isethionates, mention may be made of acylisethionates, for instance sodium cocoyl-isethionate, such as the product sold under the name Jordapon CI P® by the company Jordan.

As taurates, mention may be made of the sodium salt of palm kernel oil methyltaurate sold under the name Hostapon CT Paste® by the company Clariant; N-acyl N-methyltaurates, for instance the sodium N-cocoyl N-methyltaurate sold under the name Hostapon LT-SF® by the company Clariant or sold under the name Nikkol CMT-30-T® by the company Nikkol, and the sodium palmitoyl methyltaurate sold under the name Nikkol PMT® by the company Nikkol.

As sulfosuccinates, mention may, for example, by made of the oxyethylenated (3 EO) lauryl alcohol monosulfosuccinate (70/30 C12/C14) sold under the names Setacin 103 Special® and Rewopol SB-FA 30 K 4® by the company Witco, the disodium salt of a C12-C14 alcohol hemisulfosuccinate sold under the name Setacin F Special Paste® by the company Zschimmer Schwarz, the oxyethylenated (2 EO) disodium oleamidosulfosuccinate sold under the name Standapol SH 135® by the company Cognis, the oxyethylenated (5 EO) laurylamide mono-sulfosuccinate sold under the name Lebon A-5000® by the company Sanyo, the oxyethylenated (10 EO) disodium salt of laurylcitrate monosulfosuccinate sold under the name Rewopol SB CS 50® by the company Witco, and the ricinoleic monoethanolamide monosulfosuccinate sold under the name Rewoderm S 1333® by the company Witco.

As phosphates and alkyl phosphates, mention may, for example, be made of monoalkyl phosphates and dialkyl phosphates, such as the lauryl monophosphate sold under the name MAP 20® by the company Kao Chemicals, the potassium salt of dodecylphosphoric acid, as a mixture of monoester and diester (mainly diester) sold under the name Crafol AP-31® by the company Cognis, the mixture of octylphosphoric acid monoester and diester sold under the name Crafol AP-20® by the company Cognis, the mixture of ethoxylated (7 mol of EO) 2-butyloctanol phosphoric acid monoester and diester, sold under the name Isofol 12 7 EO-Phosphate Ester® by the company Condea, the potassium or triethanolamine salt of (C12-C13) monoalkyl phosphate sold under the references Arlatone MAP 230K-40® and Arlatone MAP 230T-60® by the company Uniqema, and the potassium lauryl phosphate sold under the name Dermalcare MAP XC-99/09® by the company Rhodia Chimie.

The polypeptides are obtained, for example, by condensing a fatty chain onto cereal, and in particular wheat and oat, amino acids. As polypeptides, mention may, for example, be made of the potassium salt of hydrolyzed lauroyl wheat protein sold under the name Aminofoam W OR® by the company Croda, the triethanolamine salt of hydrolyzed cocoyl soya bean protein sold under the name May-Tein SY® by the company Maybrook, the sodium salt of oat lauroyl amino acids, sold under the name Proteol Oat® by the company Seppic, collagen hydrolysate grafted onto coconut fatty acid, sold under the name Geliderm 3000® by the company Deutsche Gelatine, soya bean proteins acylated with hydrogenated coconut acid, sold under the name Proteol VS 22® by the company Seppic.

The anionic alkyl polyglucoside derivatives may in particular be glyceryl citrates, tartrates, sulfosuccinates, carbonates and ethers obtained from alkyl polyglucosides. Mention may, for example, be made of the sodium salt of cocoylpolyglucoside (1,4) tartaric ester, sold under the name Eucarol AGE-ET® by the company Cesalpinia, the disodium salt of cocoylpolyglucoside (1,4) sulfosuccinic ester, sold under the name Essai 512 MP® by the company Seppic, the sodium salt of cocoylpolyglucoside (1,4) citric ester sold under the name Eucarol AGE-EC® by the company Cesalpinia.

The fatty acid soaps that can be used as anionic surfactants are fatty acids of natural or synthetic origin, salified with an inorganic or organic base. The fatty chain may contain from 6 to 22 carbon atoms, preferably from 8 to 18 carbon atoms. The inorganic or organic base may be chosen from alkali metals or alkaline earth metals, amino acids and amino alcohols. As salts, use may, for example, be made of sodium salts, potassium salts, magnesium salts, triethanolamine salts, N-methylglucamine salts, lysine salts and arginine salts. As soaps, mention may, for example, be made of potassium or sodium salts of lauric acid, myristic acid, palmitic acid and stearic acid (potassium or sodium laurate, myristate, palmitate and stearate), and mixtures thereof.

3. Amphoteric and Zwitterionic Surfactants

These surfactants can be chosen, for example, from betaines, N-alkylamidobetaines and derivatives thereof, glycine derivatives, sultaines, alkyl polyaminocarboxylates and alkyl amphoacetates, and mixtures thereof.

As betaines, mention may, for example, be made of cocobetaine, for instance the product sold under the name Dehyton AB-30® by the company Cognis, laurylbetaine, for instance the product sold under the name Genagen KB® by the company Clariant, oxyethylenated (10 EO) laurylbetaine, for instance the product sold under the name Lauryl Ether (10 EO) Betaine® by the company Shin Nihon Rica, oxyethylenated (10 EO) stearylbetaine, for instance the product sold under the name Stearyl Ether (10 EO) Betaine® by the company Shin Nihon Rica.

Among the N-alkylamidobetaines and derivatives thereof, mention may, for example, be made of the cocamidopropylbetaine sold under the name Lebon 2000 HG® by the company Sanyo, or sold under the name Empigen BB® by the company Albright & Wilson, the lauramidopropylbetaine sold under the name Rewoteric AMB12P® by the company Witco.

As glycine derivatives, mention may be made of the sodium N-cocoylglycinate sold under the name Amilite GCS-12® by the company Ajinomoto.

As sultaines, mention may be made of the cocoylamidopropylhydroxysulfobetaine sold under the name Crosultaine C-50® by the company Croda.

As alkyl polyaminocarboxylates (APACs), mention may be made of the sodium cocoylpolyaminocarboxylate sold under the name Ampholak 7 CX/C® and Ampholak 7 CX® by the company Akzo Nobel, the sodium stearylpolyamidocarboxylate sold under the name Ampholak 7 TX/C by the company Akzo Nobel, the sodium carboxymethyloleylpolypropylamine sold under the name Ampholak XO7/C® by the company Akzo Nobel.

As alkyl amphoacetates, mention may, for example, be made of N-disodium N-cocoyl-N-carboxymethoxyethyl-N-carboxymethylethylenediamine (INCI name: disodium cocoamphodiacetate), for instance the product sold under the name Miranol C2M Concentrate NP® by the company Rhodia Chimie, and N-sodium N-cocoyl-N-hydroxyethyl-N-carboxymethylethylenediamine (INCI name: sodium cocoamphoacetate).

Among the surfactants mentioned above, according to a particular embodiment of the invention, use is more particularly made of one or more detergent surfactants chosen from alkyl polyglucosides, alkyl ether sulphates, N-acylglutamates, acylisethionates, sulfosuccinates, phosphates and alkyl phosphates, betaines and alkyl amphoacetates, and mixtures thereof.

The detergent surfactants are generally introduced into the aqueous phase.

The composition according to the invention is not limited with regard to pH but may generally have a pH ranging from 3 to 9, better still from 4 to 9, and better still from 4.5 to 8, and even better still from 5.5 to 8. If need be, the pH of the composition is preferably adjusted by adding an acid such as citric acid or a base such as sodium hydroxide or triethanolamine.

The compositions of the invention can constitute in particular cleansing or makeup-removing cosmetic compositions. They are preferably not foaming compositions and are not necessarily rinsed off.

The compositions of the invention may be more or less fluid and they may constitute in particular a milk or a cream, the latter being more or less thick.

Thus, according to one of the particular embodiments of the invention, the compositions are in the form of a milk, i.e. of a composition that is quite fluid so as to be able to flow out of a bottle under its own weight. The term “fluid composition” or “milk” is intended to mean a composition having a viscosity ranging, for example, from 1 to 500 cP, i.e. 0.001 Pas to 0.5 Pas, and better still ranging from 1 to 100 cP (i.e. from 0.001 to 0.1 Pa·s), this viscosity being measured at ambient temperature (approximately 25° C.) using a rheomat RM180. The milks can be used directly on the skin, but most commonly by applying a small amount of milk to a piece of cotton wool or any other appropriate support, and transferring it from the cotton wool to the skin.

According to another embodiment of the invention, the compositions are in the form of a cream, which corresponds to a texture that is thicker than a milk. Creams do not flow under their own weight and are generally held in a jar, whereas milks are held in bottles. The creams may, for example, have a viscosity at 25° C. ranging from approximately 10 to 150 poises (1 to 15 Pa·s), and preferably from approximately 15 to 100 poises (1.5 to 10 Pa·s), this viscosity being measured with a rheomat 180 device at 25° C. with the appropriate spindles (3 or 4 according to the viscosity).

The creams obtained according to the invention have the advantage of being able to contain a large amount of oil and of having a soft and unctuous texture, and of therefore being very pleasant to use. Preferably, such creams are in a jar and are used in the following way: a knob of cream is taken and is applied, with the fingers, to the dry made-up face. By massaging, the cream changes into an oil and dissolves the make-up. The face is then wiped with a dry or moist wipe such as a paper handkerchief or tissue of the Kleenex® type. The skin is cleansed without being aggressively treated; it is also nourished and moisturized, and the product leaves a soft and non-greasy skin. The advantages of these creams are that they have a dense and rich consistency, they have an unctuous aspect, they are easy to take up from the jar, they have qualities of freshness on application, and they remove makeup efficiently.

The compositions of the invention may also contain one or more adjuvants chosen from adjuvants that are used in the cosmetics field, such as hydrophilic or lipophilic active agents, preserving agents, antioxidants, fragrances, solvents, fillers, fibres, sunscreens, dyestuffs (pigments or pearlescent agents), pH-adjusting agents (bases such as sodium hydroxide or acids such as citric acid), lipid vesicles, or polymers other than those indicated above, and mixtures thereof. These adjuvants are used in the proportions that are usual in the cosmetics domain, for example from 0.01 to 20% of the total weight of the composition, and, according to their nature, they are introduced into the aqueous phase or into the oily phase of the emulsion, or else into vesicles. These adjuvants and also the concentrations thereof must be such that they do not modify the property desired for the composition.

According to the fluidity of the composition that it is desired to obtain, one or more gelling agents, in particular hydrophilic gelling agents, i.e. water-soluble or water-dispersible gelling agents, can be incorporated into the composition. As hydrophilic gelling agents, mention may in particular be made of water-soluble or water-dispersible thickening polymers. The latter may in particular be chosen from: carboxyvinyl polymers, such as the products sold under the names Carbopol (INCI name: carbomer); carboxyvinyl polymers comprising at least one hydrophobic group, such as those sold under the names Pemulen TR1 or Pemulen TR2 or Carbopol 1342 (INCI name: acrylate/C10-C30 alkyl acrylate crosspolymer) by the company Noveon; polyacrylates and polymethylacrylates, such as the products sold under the names Lubrajel and Norgel by the company Guardian or under the name Hispagel by the company Hispano Chimica, or crosslinked sodium polyacrylates such as the product sold by the company Cognis under the name Cosmedia SP; polyacrylamides; optionally crosslinked and/or neutralized 2-acrylamido-2-methanepropanesulfonic acid polymers and copolymers, such as the poly(2-acrylamido-2-methylpropane sulfonic acid) sold by the company Clariant under the name Hostacerin AMPS (INCI name: ammonium polyacryldimethyltauramide); crosslinked anionic copolymers of acrylamide and of 2-acrylamido-2-methylpropanesulfonic acid (AMPS), in the form of a W/O emulsion, such as those sold under the name Sepigel 305 (INCI name: polyacrylamide/C13-14 isoparaffin/laureth-7) and under the name Simulgel 600 (INCI name: acrylamide/sodium acryloyldimethyltaurate copolymer/isohexadecane/polysorbate 80) by the company Seppic; polysaccharides such as xanthan gum, guar gum, carob gum, acacia gum, scleroglucans, derivatives of chitin and of chitosan, carrageenans, gellans, alginates, celluloses such as microcrystalline cellulose, carboxymethylcellulose, hydroxymethylcellulose and hydroxypropylcellulose; and mixtures thereof. The amount of gelling agents depends on the desired aim. The amount of gelling agents may range, for example, from 0.01 to 10%, preferably from 0.05 to 5% by weight, and better still from 0.05 to 3% by weight, relative to the total weight of the composition.

According to a preferred embodiment of the invention, the composition contains at least one polymer chosen from carboxyvinyl polymers (carbomer), carboxyvinyl polymers comprising at least one hydrophobic group (modified carboxyvinyl polymers) (acrylate/C10-C30 alkyl acrylate crosspolymer), polysaccharides (in particular xanthan gum), crosslinked sodium polyacrylates, 2-acrylamido-2-methylpropanesulfonic acid copolymers, and blends thereof.

As active agents that can be used in the composition of the invention, mention may in particular be made of antiseborrheic and antimicrobial active agents, that in particular make it possible to treat greasy skin. This active agent may in particular be chosen from beta-lactam derivatives, quinolone derivatives, ciprofloxacine, norfloxacine, tetracycline and its salts, erythromycin and its salts, amikacin and its salts, 2,4,4′-trichloro-2′-hydroxydiphenyl ether (or triclosan), 3,4,4′-trichlorocarbanilide (or triclocarban), phenoxyethanol, phenoxypropanol, phenoxyisopropanol, doxycycline and its salts, capreomycin and its salts, chlorhexidine and its salts, chlortetracycline and its salts, oxytetracycline and its salts, clindamycin and its salts, ethambutol and its salts, hexamidine isethionate, metronidazole and its salts, pentamidine and its salts, gentamycin and its salts, kanamycin and its salts, lincomycin and its salts, methacycline and its salts, methenamine and its salts, minocycline and its salts, neomycin and its salts, netilmicin and its salts, paromomycin and its salts, streptomycin and its salts, tobramycin and its salts, miconazole and its salts, amantadine salts, para-chloro-meta-xylenol, nystatin, tolnaftate, salicylic acid and its salts, 5-n-octanoyl salicylic acid and its salts, benzoyl peroxide, 3-hydroxybenzoic acid, 4-hydroxybenzoic acid, acetylsalicylic acid, 2-hydroxybutanoic acid, 2-hydroxypentanoic acid, 2-hydroxyhexanoic acid, phytic acid, N-acetyl-L-cysteine, lipoic acid, azelaic acid, arachidonic acid, ibuprofen, naproxene, hydrocortisone, acetaminophen, resorcinol, octopirox, lidocaine hydrochloride, clotrimazole, octoxyglycerine, octanoylglycine, caprylyl glycol, 10-hydroxy-2-decanoic acid, zinc salts such as zinc gluconate, niacinamide or vitamin B3 (or vitamin PP), and mixtures thereof.

As other active agents that can be used in the composition of the invention, mention may also be made of keratolytic and anti-ageing agents, for instance hydroxy acids (α-hydroxy acids and β-hydroxy acids) such as lactic acid, glycolic acid, citric acid, salicylic acid and derivatives thereof; essential oils; vitamins, and in particular retinol (vitamin A), ascorbic acid (vitamin C), tocopherol (vitamin E), niacinamide (vitamin PP or B3), panthenol (vitamin B5) and derivatives thereof (esters, for example); coenzymes, and in particular coenzyme Q10 or ubiquinone; enzymes such as, for example, lipases, proteases, phospholipases, cellulases, peroxydases, especially lactoperoxydases, catalases, superoxyde dismutases and plant extracts containing the abovementioned enzymes; yeast such as Saccharomyces cerevisiae; steroids; antioxidants and free-radical scavengers; moisturizers such as polyols (glycerol, sorbitol, sugars), protein hydrolysates, urea and mixtures containing them; slimming agents such as caffeine; anti-elastase and anti-collagenase agents; agents for treating greasy skin; plant extracts, and in particular plankton extracts; tensioning agents; matifying agents, antiperspirants such as aluminium salts; and mixtures thereof.

As salicylic acid derivatives, mention may in particular be made of 5-n-octanoylsalicylic acid (INCI name: capryloyl salicylic acid), S-n-decanoylsalicylic acid, 5-n-dodecanoylsalicylic acid, 5-n-octylsalicylic acid, 5-n-heptyloxysalicylic acid, 5-tert-octylsalicylic acid, 5-butoxysalicylic acid, 5-ethoxysalicylic acid, 5-methoxysalicylic acid, 5-propoxysalicylic acid, 5-methylsalicylic acid, 5-ethylsalicylic acid and 5-propylsalicylic acid, and mixtures thereof. These acids may optionally be salified with a base.

As examples of steroids, mention may, for example, be made of dehydroepiandrosterone (or DHEA), and also (1) its biological precursors and derivatives, in particular DHEA salts and esters, such as DHEA salicylate and sulphate, 7-hydroxy DHEA, 7-keto DHEA, 7-hydroxy and 7-keto DHEA esters, in particular 3-beta-acetoxy-7-oxo DHEA, and (2) its chemical precursors and derivatives, in particular sapogenins such as diosgenin or hecogenin, and/or their derivatives such as hecogenin acetate, and/or natural extracts containing the same, and in particular extracts of Dioscoreae, such as wild yam.

The composition of the invention may also contain fillers with a view to modifying the texture of the composition. As fillers that may be used in the composition of the invention, mention may, for example, be made, in addition to pigments, of silica; talc; polyamide particles, and in particular those sold under the name Orgasol by the company Atochem; polyethylene powders; acrylic copolymer-based microspheres, such as those made of ethylene glycol dimethacrylate/lauryl methacrylate copolymer sold by the company Dow Corning under the name Polytrap; expanded powders such as hollow microspheres, and in particular the microspheres sold under the name Expancel by the company Kemanord Plast or under the name Micropearl F 80 ED by the company Matsumoto; powders of natural organic materials, such as crosslinked or non-crosslinked maize, wheat or rice starches, such as the powders of starch crosslinked with octenylsuccinic anhydride, sold under the name Dry-Flo by the company Natinal Starch; silicone resin microbeads such as those sold under the name Tospearl by the company Toshiba Silicone; and mixtures thereof. These fillers may be present in amounts ranging from 0 to 20% by weight, and preferably from 1 to 10% by weight, relative to the total weight of the composition.

Of course, those skilled in the art would take care to choose the optional compound(s) to be added to the composition according to the invention in such a way that the advantageous properties intrinsically associated with the composition in accordance with the invention are not, or are not substantially, impaired by the envisaged addition.

The compositions of the invention can constitute in particular makeup-removing and/or cleansing products for the skin, the lips and/or the eyes.

A subject of the invention is also the cosmetic use of the composition as defined above, for removing makeup from and/or cleansing the skin, the lips and/or the eyes.

The composition of the invention can most particularly be used for makeup removal or cleansing.

Thus, a subject of the present invention is also a process for removing makeup from and/or for cleansing the skin, the lips and/or the eyes, wherein a composition as defined above is applied to the skin, the lips and/or the eyes.

Moreover, when the compositions according to the invention have an appropriate fluidity and are, for example, in the form of a milk, they can also be used to impregnate water-insoluble substrates so as to constitute articles (such as wipes) for use in cleansing and/or removing makeup from the skin, the eyelashes and/or the lips. The water-insoluble substrate may comprise one or more layers and it may be chosen from the group comprising woven materials, nonwoven materials, foams, sponges, wadding, felt, balls or films. It may in particular be a nonwoven substrate based on fibres of natural origin (flax, wool, cotton or silk) or of synthetic origin (cellulose derivatives, viscose, polyvinyl derivatives, polyesters such as polyethylene terephthalate, polyolefins such as polyethylene or polypropylene, polyamides such as nylon, and acrylic derivatives). The nonwovens are described in general in Riedel's “Nonwoven Bonding Methods & Materials”, Nonwoven World (1987). These substrates are obtained according to the usual processes of the technique for preparing nonwovens.

When the substrate is a nonwoven, a thick nonwoven is preferably used, which does not roll up into a ball and which is solid enough not to disintegrate and not to pill when applied to the skin. It should be absorbent and soft on at least one face for removing makeup in particular from the eyes. As suitable nonwovens, mention may, for example, be made of those sold under the names Ultraloft 15285-01, Ultraloft 182-008, Ultraloft 182-010 and Ultraloft 182-016 by the company BBA, Vilmed M1519 Blau, Vilmed M 1550 N and 112-132-3 by the company Freudenberg, the product sold under the name Norafin 11601-010B by the company Jacob Holm Industries, and the flocked nonwovens sold under the names Univel 109 and Univel 119 by the company Uni Flockage.

Moreover, this substrate may comprise one or more layers having identical or different properties and having elasticity and softness properties and other properties suitable for the desired use. The substrates may comprise, for example, two parts having different elasticity properties, as described in document WO-A-99/13861, or may comprise a single layer with different densities, as described in document WO-A-99/25318 or may comprise two layers of different textures as described in document WO-A-98/18441.

A subject of the invention is therefore also an article obtained by impregnating a water-insoluble substrate with a composition as defined above.

A subject of the invention is also the use of the composition as defined above, for preparing an article intended for removing makeup from or cleansing the skin, the lips and/or the eyelashes.

The following examples of compositions according to the invention are given by way of illustration that is in no way limiting in nature. The amounts therein are given as % by weight, unless otherwise mentioned.

### EXAMPLE 1

### According to the Invention: Makeup-Removing Milk

| Phase A | |
|---|---|
| glycerol | 3% |
| preserving agents | 0.2% |
| dimethicone copolyol (DC 5225 C) | 1.5% |
| (i.e. 0.15% of active material) | |
| disodium cocoamphodiacetate | 1% |
| sodium laureth sulphate | 1% |
| pemulen TR2 | 0.15% |
| water | qs 100% |
| Phase B | |
| ethylhexyl palmitate | 15% |
| Phase C | |
| sodium hydroxide (NaOH) | qs pH 6.5 |
| water | 5% |

Procedure: Phase A was prepared by heating the mixture of compounds, without the pemulen, to 65° C.-70° C., the pemulen was then added by dispersing it with shear, and phase B was subsequently added, with agitation, followed by phase C.

A smooth and shiny, homogeneous soft white milk was obtained, which was pleasant to use and whose makeup-removing efficiency was good.

### COMPARATIVE EXAMPLE 2

| Phase A | |
|---|---|
| glycerol | 3% |
| preserving agents | 0.2% |
| disodium cocoamphodiacetate | 1% |
| sodium laureth sulphate | 1% |
| pemulen TR2 | 0.15% |
| water | qs 100% |
| Phase B | |
| ethylhexyl palmitate | 15% |
| Phase C | |
| sodium hydroxide (NaOH) | qs pH 6.5 |
| water | 5% |

Procedure: As in Example 1. A milk which was soft and homogeneous, but whose makeup-removing efficiency was not good, is obtained.

### COMPARATIVE EXAMPLE 3

| Phase A | |
|---|---|
| glycerol | 5% |
| preserving agents | 0.2% |
| disodium cocoamphodiacetate | 1% |
| sodium laureth sulphate | 1% |
| xanthan gum | 0.1% |
| carbomer | 0.5% |
| water | qs 100% |
| Phase B | |
| liquid petroleum jelly | 12% |
| ethylhexyl palmitate | 7% |
| cetearyl alcohol | 0.4% |
| Phase C | |
| sodium hydroxide (NaOH) | qs pH 6.5 |
| water | 5% |

Procedure: as in Example 1. A milk which was smooth and soft, but whose makeup-removing efficiency was not good, is obtained.

Makeup-removing test: A test was carried out to compare the efficiency of makeup-removal of Example 1 according to the invention, with respect to Comparative Examples 2 and 3.

I. In Vivo Makeup-Removing Protocol

I.a. Material

1. Makeup-removing oil that can be rinsed off with water: “Huile Démaquillante fraîche” [fresh makeup-removing oil] from Shu Uemura.
2. Household soap.
3. 1 masking card comprising an empty central portion cut out in a 4×4 cm Bristol board+1 Lumicolor permanent S marker pen.
4. CR300 colorimeter (L.a.b. colorimetry measurement).
5. Air Wear no-transfer foundation “Sable” LSF 12 from L'Oreal.
6. 8 small watch glasses+small spatulas.
7. 1 precision balance.
8. 1 stopwatch.
   I.b Protocol
   4 areas (2 per arm), the size of the central portion cut out of the masking card, are drawn on the skin using the marker pen and the masking card;
   the skin in these areas is cleansed with the rinsable makeup-removing oil and then with the soap, rinsed and dried (the marks from the marker pen are therefore lightened so as to prevent any transfer of marker ink to the foundation at the time of application);
   the naked skin is measured by colorimetry: 3 measurements of L,a,b per area (calorimeter reading head positioned right at the centre of the area);
   16 mg+/−0.05 mg of foundation are applied evenly in the 4 delimited areas, and left for 30 minutes;
   the skin with foundation is measured by colorimetry: 3 measurements of L,a,b per area;
   208 mg+/−1 mg of makeup-removing composition to be tested are weighed out into the watch glasses 4 times;
   the makeup is removed from each area with the finger by circular movement for 10 seconds and the area is then rinsed with tap water (warm; hardness not controlled), lightly brushing with the fingers of the available hand. This is done to the 4 areas without waiting;
   the areas are dabbed with a kleenex-type disposable handkerchief and left for 15 minutes (feeling that the skin is dry);
   the skin from which the makeup has been removed is measured by colorimetry: 3 measurements of L,a,b per area.

The percentage makeup removal is calculated in the following way:

the calorimetric difference in the made-up-skin (FdT skin) relative to the naked skin is determined, ΔEmax being the value corresponding to complete makeup removal:
ΔE max=√(Δa₁²+Δb₁²+ΔL₁²) with
Δa₁=a naked skin−a FdT skin
Δb₁=b naked skin−b FdT skin
ΔL₁=L naked skin−L Fdt skin

for the makeup-removing composition tested, the colorimetric difference ΔE of the skin from which the makeup has been removed (remove skin) relative to the made-up skin (FdT skin) is determined for each area:
ΔE=(Δa₂+Δb₂²+ΔL₂²) with
Δa₂=a remove skin−a FdT skin
Δb₂=b remove skin−b FdT skin

### ΔL₂=L remove skin−L FdT skin

The ΔE/ΔEmax ratio is determined.

The mean makeup removal percentage, which is the mean of the 4 values of (ΔE/ΔEmax)×100, is calculated for the makeup-removing composition tested.

The following table gives the results of Example 1 of the invention and of Comparative Examples 2 and 3:

| Makeup-removing | Amount of makeup- | Makeup-removing |
|---|---|---|
| composition | removing oil | efficiency |
| Example 1 | 15% | 43% |
| according to the | | |
| invention | | |
| Comparative | 15% | 26% |
| Example 2 | | |
| Comparative | 19% | 19% |
| Example 3 | | |

Comparison of Examples 1 and 3 shows that the makeup-removing efficiency was better for Example 1 according to the invention, which nevertheless contains less oil than the Comparative Example 3.

Comparison of Examples 1 and 2 shows that the makeup-removing efficiency was better for Example 1, which differs from Example 2 only through the addition of 1.5% of DC 5225 C.

### EXAMPLE 4

### According to the Invention: Makeup-Removing Cream

| Phase A | |
|---|---|
| glycerol | 3% |
| preserving agents | 0.3% |
| disodium cocoamphodiacetate | 0.75% |
| sodium laureth sulphate | 0.75% |
| water | qs 100% |
| Phase B | |
| ethylhexyl palmitate | 20% |
| liquid petroleum jelly | 15% |
| petrolatum | 20% |
| Phase C | |
| liquid petroleum jelly | 5% |
| carbomer | 0.7% |
| Phase D | |
| dimethicone copolyol (DC 5225 C) | 0.7% |
| (i.e. 0.07% of active material) | |
| Phase E | |
| water | 1.5% |
| triethanolamine | 0.8% |

Procedure: Phase A was heated to 75° C., then phase B followed by phase C were added to phase A, with agitation, so as to form the emulsion. Phase D was subsequently added with agitation. Finally, the mixture was neutralized by adding phase E.

A cream having a viscosity of approximately 40 poises at 25° C. was obtained. It was a smooth, shiny and unctuous white cream, which can be efficiently used for removing makeup from the skin.

The above written description of the invention provides a manner and process of making and using it such that any person skilled in this art is enabled to make and use the same, this enablement being provided in particular for the subject matter of the appended claims, which make up a part of the original description and including a cosmetic composition in the form of an oil-in-water emulsion, containing an oily phase dispersed in an aqueous phase, wherein it comprises at least one silicone surfactant, at least one oil and at least one detergent surfactant.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition in the form of an oil-in-water emulsion, comprising an oily phase dispersed in an aqueous phase, said composition comprising at least one silicone surfactant, at least one oil, and at least one detergent surfactant.

2. The composition according to claim 1, wherein the silicone surfactant is chosen from dimethicone copolyols, alkyl dimethicone copolyols, and mixtures thereof.

3. The composition according to claim 1, wherein the silicone surfactant comprises from 2 to 50 oxyethylenated groups and from 2 to 50 oxypropylenated groups.

4. The composition according to claim 1, wherein the silicone surfactant is chosen from dimethicone copolyol comprising 18 oxyethylenated groups and 18 oxypropylenated groups, dimethicone copolyol comprising 14 oxyethylenated groups and 14 oxypropylenated groups, cetyl dimethicone copolyol, lauryl dimethicone copolyol, and mixtures thereof.

5. The composition according to claim 1, wherein the amount of silicone surfactant is 0.01 to 5% by weight relative to the total weight of the composition.

6. The composition according to claim 1, wherein the amount of oily phase is 3 to 70% by weight relative to the total weight of the composition.

7. The composition according to claim 1, comprising at least one makeup-removing oil chosen from hydrocarbon-based oils, fatty esters comprising at least 8 carbon atoms, and mixtures thereof.

8. The composition according to claim 1, wherein the amount of detergent surfactant(s) is 0.05 to 10% by weight relative to the total weight of the composition.

9. The composition according to claim 1, wherein the at least one detergent surfactant has an HLB greater than or equal to 11.

10. The composition according to claim 1, wherein the detergent surfactant is chosen from nonionic, anionic, amphoteric and zwitterionic detergent surfactants, and mixtures thereof.

11. The composition according to claim 1, wherein the detergent surfactant is chosen from alkyl polyglucosides, alkyl ether sulphates, N-acylglutamates, acylisethionates, sulphosuccinates, phosphates and alkyl phosphates, betaines and alkyl amphoacetates, and mixtures thereof.

12. The composition according to claim 1, further comprising at least one polymer chosen from carboxyvinyl polymers, carboxyvinyl polymers comprising at least one hydrophobic group, polysaccharides, crosslinked sodium polyacrylates, 2-acrylamido-2-methylpropanesulphonic acid polymers, and mixtures thereof.

13. The composition according to claim 1, wherein the composition comprises no other emulsifier other than the at least one silicone surfactant.

14. The composition according to claim 2, wherein the composition comprises no other emulsifier other than the at least one silicone surfactant.

15. The composition according to claim 3, wherein the composition comprises no other emulsifier other than the at least one silicone surfactant.

16. The composition according to claim 4, wherein the composition comprises no other emulsifier other than the at least one silicone surfactant.

17. A process, comprising applying to the skin, the lips and/or the eyes a composition according to claim 1.

18. A process, comprising applying to the skin, the lips and/or the eyes a composition according to claim 16.

19. An article comprising a water-insoluble substrate impregnated with a composition according to claim 1.

20. An article comprising a water-insoluble substrate impregnated with a composition according to claim 16.
